(19) United States
(12) Patent Application Publication
(10) Pub. No.: US 2014/0220133 A1
(43) Pub. Date: Aug. 7, 2014

(54) Nanotechnology Based Medicine for Biodefense

(71) Applicant: CytImmune Sciences, Inc., Rockville, MD (US)

(72) Inventors: Giulio F. Paciotti, Ellicott City, MD (US); Lawrence Tamarkin, Rockville, MD (US)

(21) Appl. No.: 14/092,338

(22) Filed: Nov. 27, 2013

Related U.S. Application Data
(60) Provisional application No. 61/730,844, filed on Nov. 28, 2012.

(51) Int. Cl. A61K 39/44; A61K 47/48

(52) U.S. Cl. CPC A61K 39/44; A61K 47/48646; A61K 47/4853
USPC 424/489; 424/142.1

(57) ABSTRACT

A composition and methods to bind and remove toxic agents from a subject exposed to the toxic agents are described herein. The composition comprises a stealth agent and scavenging agent bound to a nanoparticle platform. The stealth agent prevents the nanomedicine from detection and elimination by the immune system allowing the scavenging agent to bind the target toxic agent. The stealth agent comprises an exposing group that once removed from the stealth agent allows the nanomedicine and bound toxic agent to be detected and eliminated from the subject's body.

### CROSS REFERENCE TO RELATED APPLICATIONS

This application claims the benefit of U.S. Provisional Patent Application No. 61/730,844 filed Nov. 28, 2012.

### FIELD OF THE INVENTION

The present invention relates nanoparticle-based medicines for use in capturing and eliminating biological toxic agents. The nanoparticle-base medicines comprise a scavenging agent for binding biological toxins and a stealth agent that provides the nanoparticle-based medicine increased residence times in the blood. Upon release of an exposing group from the stealth agent the nanomedicine is immunologically recognized and the particle and the biological toxins bound to it are cleared by the immunological system.

### BACKGROUND OF THE INVENTION

The goal of any bioterrorist attack is to inflict injury and more importantly to cause societal paralysis. In effect it is widely believed that the psychological impact of a bioterrorist attack may be more crippling to a nation's security than the attack itself. Compounding those threats is a real absence of effective therapies that not only negate the clinical manifestations of the toxic agent used in the biological weapon, but also reassure citizens of their well-being. Consequently, development of readily deployable/cost-effective countermeasures for both civilian and military responses to these threats remains a top priority in homeland security and national defense.

### SUMMARY OF INVENTION

In certain example embodiments described herein, a nanoparticle-based composition for capturing and eliminating biological toxic agents from a subject's body is provided, the composition comprising a scavenging agent specific for a biological toxin and a bi-functional stealth agent bound to a nanoparticle platform. In one example embodiment, the bi-functional stealth agent comprises a nanoparticle-binding group, a core molecule and an exposing group. In another example embodiment, the bi-functional stealth agent comprises a nanoparticle-binding group, a core molecule, a cleavable linker, and an exposing group.

The nanoparticle platform may comprise such materials as, but not limited to, gold nanoparticles, silver nanoparticles, silica nanoparticles, iron nanoparticles, metal hybrid nanoparticles such as gold/iron nanoparticles, nanoshells, gold nanoshells, silver nanoshells, gold nanorods, silver nanorods, metal hybrid nanorods, quantum dots, nanoclusters, liposomes, dendrimers, metal-liposome particles, metal-dendrimer nanohybrids and carbon nanotubes. In one example embodiment, the nanoparticle platform is a colloidal gold nanoparticle.

The scavenging agent may bind the pathogen or a component of the pathogen. In one example embodiment, the scavenging agent binds a toxin produced by a pathogen, such as Botulinum toxin. In another example embodiment, the scavenging agent binds a bacterial wall or viral coat protein, such as a Dengue virus coat protein. In one example embodiment the scavenging agent is a monoclonal antibody. In another example embodiment, the scavenging agent is a human monoclonal antibody. In yet another example embodiment, the scavenging agent is the Fab fragment of a human monoclonal antibody.

In another example embodiment, methods for binding and removing pathogens is provided comprising administering the above composition to subjects in need therefore. In one example embodiment, the composition is administered to a subject that has been exposed to a biological weapon. In another example embodiment, the composition is administered in an extended release formulation. For example, the composition may be encapsulated in or bound to a microparticle.

These and other aspects, objects, features, and advantages of the example embodiments will become apparent to those having ordinary skill in the art upon consideration of the following detailed description of the example embodiments.

### BRIEF DESCRIPTION OF THE DRAWINGS

FIG. 1 is a diagram of a nanomedicine according to an example embodiment.

FIG. 2 is diagram showing shedding of a stealth agent exposing group and subsequent routes of detection by the immunological system according to example embodiments.

FIG. 3 is a transmission electron micrograph showing colloidal nanoparticles synthesized according to an example embodiment.

FIG. 4 is a graph showing particle size distribution as measured by differential disc centrifugation for 5 different batches of gold nanoparticles manufactured according to an example embodiment.

FIG. 5 is a chromatogram of PEG-THIOL fractionated by RP-HPLC and detected by evaporative light scattering detection (ELSD).

FIG. 6 is an example standard curve for a competitive ELISA showing binding of an example target biological toxin.

FIG. 7A is diagram, showing synthesis of an example stealth agent with an exposing group that will generate a free acid, in accordance with example embodiments.

FIG. 7B is a table showing structures of example stealth agents and their anticipated stability in blood in accordance with example embodiments

FIG. 8 is a table showing additional example stealth agents and their anticipated stability in blood, in accordance with example embodiments.

FIG. 9 is a schematic showing synthesis of an example stealth agent using a THIOL-PEG maleic anhydride precursor in accordance with an example embodiment.

FIG. 10 is a graph showing the predicted changes in hydrodynamic volume of an example nanomedicine following the release of an exposing agent from a THIOL-PEG² polymer.

FIG. 11 is a schematic showing an example manufacturing system for generating nanomedicines in accordance with an example embodiment.

FIG. 12 is a graph showing the anticipated impact of example nanomedicines on the pharmacokinetics of a target toxic agent.

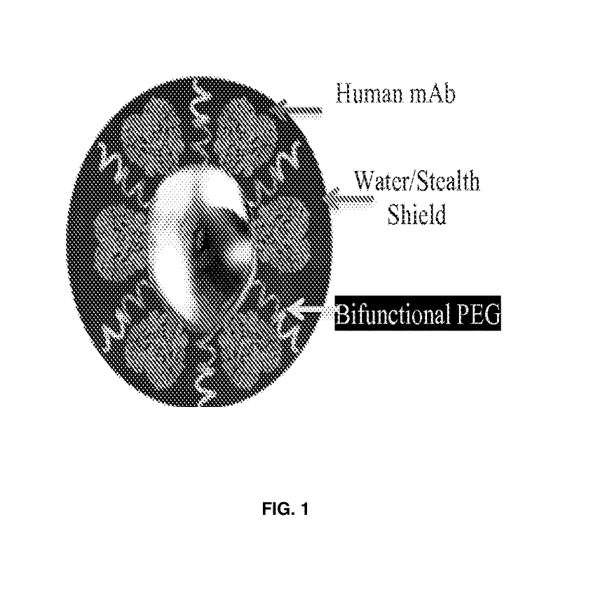

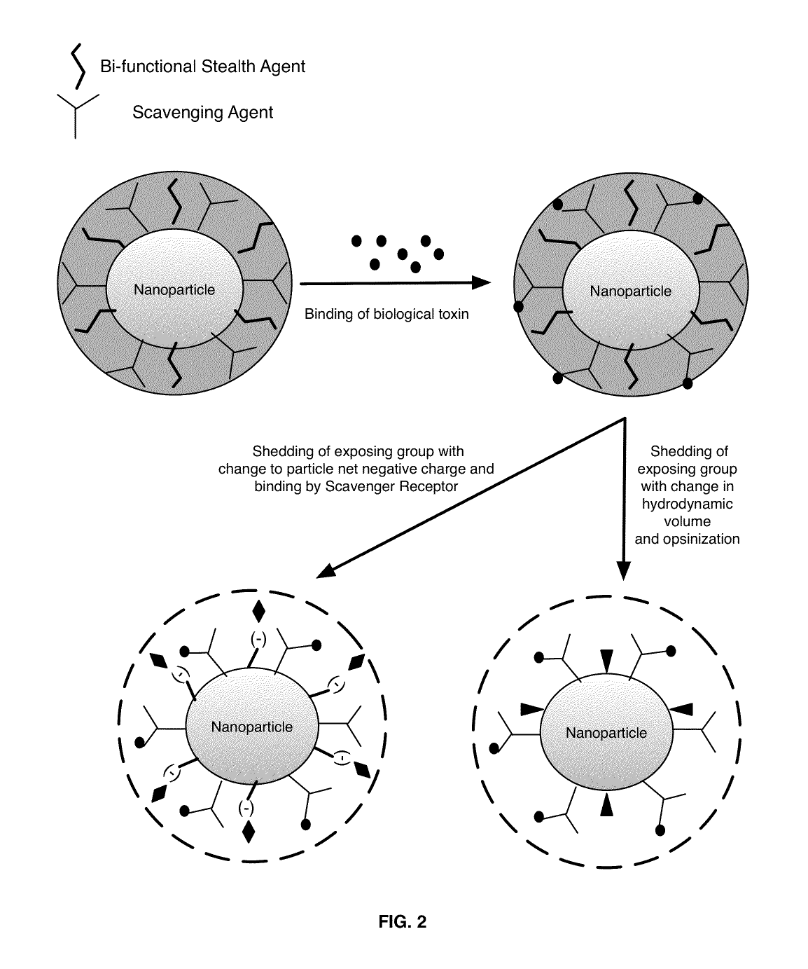

### DETAILED DESCRIPTION

The embodiments described herein provide compositions and method of use for binding and eliminating biological toxins from a subject exposed to such biological toxins. As used herein, the term “subject” includes humans and non-human animals. “Subject” and “patient” are used interchangeably herein. In particular the compositions and methods provided herein can be used to bind and eliminate biological toxins from a subject who has been exposed to the biological toxins during a biological weapon attack. The composition comprises a nanoparticle-platform, a scavenging agent bound to the nanoparticle platform, and a bi-functional stealth agent bound to the nanoparticle platform. In one example embodiment, the bi-functional stealth agent comprises a nanoparticle platform-binding group, a core molecule and an exposing group. In another example embodiment, the bi-functional stealth agent comprising a nanoparticle platform-binding group, a core molecule, a cleavable linker, and an exposing group.

Each component of the nanoparticle-based composition (“nanomedicine”) performs a function to ensure successful binding and elimination of a target biological toxin from the body. First, the bi-functional stealth agent provides the nanomedicine with immune avoidance properties that prevent immediate recognition and removal of the nanomedicine from the bloodstream by the immune system. In creating this immune avoidance shield the bi-functional stealth agent will ensure that the scavenging agent has sufficient residence time in the circulation and tissues to bind and immobilize the target biological toxin on the nanoparticle's surface and thereby prevent the clinical sequelae that would otherwise result.

Elimination of the nanomedicine-bound biological toxin from the body is triggered by physiochemical changes in the bi-functional stealth agent that results in a partial shedding of the stealth agent from the pathogen-particle complex. The detectable form of the nanomedicine is then recognized by the innate immune system in the liver and removed from circulation.

The innate immune system, also known as the monocyte phagocytic system (MPS), continuously clears particulates from the blood stream by several mechanisms, using the specialized cells present in the liver known as the Kupffer cells. These cells have evolved functions that continually sense and clear the blood of harmful pathogens. Although “immune cells” by nature, Kupffer cells have developed a mechanism termed adaptive tolerance that allows them to effectively clear particles without inducing an overwhelming immune response. To ensure that a robust response is not generated with the liver these cells continuously secrete immunosuppressive cytokines, such as interleukin-10, and immunosuppressive mediators, such as prostaglandin E₂.

One mechanism for particulate clearance utilizes a unique set of receptors known as scavenger receptors in the liver that recognize negatively charged particulates. In one example embodiment, removal of the exposing group on the bi-functional stealth agent will result in a nanomedicine obtaining a net negative charge, thereby targeting the nanomedicine particle to the scavenger receptors.

Alternatively, the MPS may clear particulates through the process of opsinization. In this process, blood-borne particulates are coated with proteins known as opsins. These opsins act as recognition signals for Kupffer cells to rapidly phagocytize the opsinized particle leading to destruction. In another example embodiment, removal of the exposing group reduces the hydrodynamic volume (“water shield”) around the nanomedicine. The loss of the water shield will allow the nanomedicine particulates to be opsinized or otherwise rapidly taken up by the MPS.

### Nanoparticle Platforms

The nanoparticle platforms of the present invention provide a base on which other molecules may be assembled to form the nanomedicines of the present invention. The nanoparticle platforms may comprise materials such as, but not limited to, colloidal metals, gold nanoparticles, silver nanoparticles, silica nanoparticles, iron nanoparticles, metal hybrid nanoparticles such as gold/iron nanoparticles, nanoshells, gold nanoshells, silver nanoshells, gold nanorods, silver nanorods, metal hybrid nanorods, quantum dots, nanoclusters, liposomes, dendrimers, metal-liposome particles, metal-dendrimer nanohybrids and carbon nanotubes. Attachment of the one or more active agents to a nanoparticle platform may slow the hydrolytic conversion of the active agents in the circulation. In certain embodiments, the attachment of the active agents to the nanoparticle platform may also control the rate at which the agents are converted to their active from. In certain embodiments where the nanoparticle platform comprises a magnetic or paramagnetic material, the nanoparticle platform may facilitate detection of the vector composition.

In one exemplary embodiment, the nanoparticle platform comprises a colloidal metal material. The colloidal metal material may include water-insoluble metal particles or metallic compounds dispersed in a liquid, a hydrosol, or a metal sol. The colloidal metal may be selected from the metals in groups IA, IB, IIB and IIIB of the periodic table, as well as the transition metals, especially those of group VIII. Preferred metals include gold, silver, aluminum, ruthenium, zinc, iron, nickel and calcium. Other suitable metals also include the following in all of their various oxidation states: lithium, sodium, magnesium, potassium, scandium, titanium, vanadium, chromium, manganese, cobalt, copper, gallium, strontium, niobium, molybdenum, palladium, indium, tin, tungsten, rhenium, platinum, and gadolinium. The metals are preferably provided in ionic form, derived from an appropriate metal compound, for example the Al³⁺, Ru³⁺, Zn²⁺, Fe³⁺, Ni²⁺ and Ca²⁺ ions.

In one exemplary embodiment, the colloidal metal is gold. In certain embodiments, the colloidal gold is in the form of Au³⁺. In another exemplary embodiment, the colloidal gold is HAuCl₄. The colloidal gold particles may have a negative charge at an approximately neutral pH. It is thought that this negative charge prevents the attraction and attachment of other negatively charged molecules. In contrast, positively charged molecules are attracted to and bind to the colloidal gold particle. The colloidal gold may be employed in the form of a sol that contains gold particles having a range of particle sizes. In one exemplary embodiment, the size of the gold particles ranges from approximately 1 to approximately 100 nm; from approximately 1 to approximately 50 nm; from approximately 1 to approximately 40 nm; from approximately 1 to approximately 30 nm; from approximately 1 to approximately 20 nm; from approximately 1 to approximately 10 nm; from approximately 20 to approximately 30 nm; from approximately 20 to approximately 50 nm; from approximately 20 to approximately 70 nm; from approximately 30 to approximately 50 nm; from approximately 30 to approximately 70 nm; from approximately 30 to approximately 90 nm; from approximately 30 to approximately 100 nm; or from approximately 50 to approximately 100 nm.

In another exemplary embodiment, the colloidal metal is colloidal silver in a sodium borate buffer, having a concentration of approximately 0.1% to approximately 0.001% of solution. In another exemplary embodiment, the colloidal silver is at a concentration of 0.01% solution in a sodium borate buffer.

### Scavenging Agent

A scavenging agent is a molecule bound to the nanoparticle surface that binds the targeted biological toxin. Of particular interest are biological toxins that can be readily weaponized and delivered as biologic weapons. A biological toxin may be a pathogen, such as a virus or bacteria, or a component or metabolic product of the pathogen. In certain example embodiments, the biological toxin is botulinum toxin, ricin toxin, epsilon toxin, Staphylococcus entertoxin B. The botulinum toxin may be the A, B, C1, D, E, F, or G form of botulinum toxin.

In certain example embodiments, the pathogen is a virus. For example, the virus may be the causative agent of Small Pox, Rabies, SARS, Hantavirus, Nipah, or Influenza. The virus may be a flaviviruses, filoviruses, or arenaviruses. Example flaviviruses include Dengue virus. Example filovirues include Ebola and Marburg viruses. Example Arenaviruses include Lassa and Machapo viruses. In certain example embodiments, the scavenging agent binds a protein of the viral coat. In one example embodiment, the virus is Dengue virus and the scavenging agent binds a viral coat protein of Dengue virus. In one example embodiment, the viral coat protein is Dengue Envelope Protein E (DENV E). In another example embodiment, the scavenging agent binds to all isoforms of a target viral protein, such as DENV E.

In certain example embodiments, the pathogen is a bacterium. For example, the bacterium may be Bacillus anthracis, Yersinia pestis, Francisellatularensi, Salmonella, E. coli, such as strain O157:H7, Shigella, Coxiellaburnetii, Rickettsia prowazekii, or the various species and strains of mycobacteria that cause Tuberculosis.

The scavenging molecule may include any molecule capable of binding to specific cells or cellular components of the above biological toxins. A cellular component includes proteins found on the outer wall or membrane of an organism, proteins contained within an organism or virus, or proteins and/or other metabolic products excreted by an organism.

In certain example embodiments, the scavenging agent is one member of a binding pair, the other member of the binding partner constituting the target biological toxin. Such selectivity may be achieved by binding to structures found naturally on cells, such as receptors found on the surface of a cellular membrane, a nuclear membrane, an organelle membrane, or a viral coat. Scavenging agents may also include receptors or parts of receptors that bind to molecules such as antibodies, antibody fragments, enzymes, cofactors, substrates, and other binding member pairs known to those of skill in the art. Scavenging agents may also be capable of binding to multiple types of binding partners. For example, the scavenging agent may bind to a class or family of toxic agents, such as all seven forms of the botulinum toxin.

In one exemplary embodiment, the targeting ligand may be a nucleic acid-based ligand. In certain exemplary embodiments, the nucleic acid-based ligand may be an aptamer. Aptamers are nucleic acids sequences that adapt a specific secondary and tertiary structure and like antibodies exhibit specific molecular recognition. Aptamers specific to any number of biological toxins can be designed using systematic evolution of ligand by exponential enrichment (SELEX) carried out in the presence of the target biological toxin. International Patent Application Publication no. WO/2009/014705 to Keefe et al. describes methods for the in vivo selection of aptamers that may be linked to therapeutic or diagnostic compositions. International Patent Application Publication No. WO/2009/090554 to Tavitian et al. describe a modified SELEX system for generating aptamers.

In another example embodiment the scavenging agent is an antibody. Suitable antibodies include monovalent and divalent single chain antibody fragments (scFv), as well as fusion protein constructs thereof. In one example embodiment, the antibody is a monoclonal antibody. In another example embodiment, the monoclonal antibody is a human monoclonal antibody. The human monocolonal antibody may be produced using the methods described in U.S. Pat. Nos. 7,960,145 and 8,486,663 to Paciotti et al., which are hereby incorporated by reference. In one example embodiment, the scavenging agent is the Fab fragment of a human monoclonal antibody.

In one example embodiment, the human monoclonal antibody binds botulinum toxin. In another example embodiment the human monoclonal antibody binds all seven forms of botulinum toxin. In one example embodiment, the human monoclonal antibody binds a Dengue virus serotype. In another example embodiment, the human monoclonal antibody binds DENV E.

### Stealth Agents

As used herein, a “stealth agent” refers to any compound which when bound to the surface of the nanoparticle platform assists in protecting the nanomedicine from being digested, absorbed, opsinized, or eliminated from the body by other metabolic activity prior to achieving its desired function.

Each stealth agent must first protect the nanomedicine from immune recognition and then permit immune recognition once the toxic agent is captured. While not bound by the following theory, it is believed the intact bi-functional stealth agent hydrates the nanoparticle by drawing twice its molecular weight in water to the surface of the nanomedicine. In this “stealth mode,” the nanomedicine circulates in the blood for a fixed period of time. Subsequently, at a fixed time after scavenging is complete, a segment of the bi-functional stealth agent is released from the nanomedicine, which in turn causes the nanomedicine with bound toxic agent to become fully detectable by at least the innate immune system.

The bi-functional stealth agent comprises at least a base molecule to which are attached a nanoparticle binding group at one terminus and an exposing group at the other terminus. The nanoparticle binding group enables the bi-functional stealth agent to bind to the surface of the nanoparticle platform. The exposing groups are releasable. In one example embodiment, the loss of the exposing group results in reduction of the hydrodynamic volume of the nanomedicine thereby allowing detection by the immune system. In another example embodiment, the loss of the exposing group results in changing the surface charge of the particle from zero or slightly positive to negative thereby allowing detection by the immune system.

Example Core Molecules

In certain exemplary embodiments, the core molecule of the stealth agent may comprise glycol compounds, preferably polyethylene glycol (PEG), (also known by those of ordinary skill in the art as polyoxyethylene or POE). The PEG may be a derivatized PEG. The present invention comprises compositions comprising derivatized PEG, wherein the PEG is 5,000 to 30,000 (daltons) MW. Derivatized PEG compounds are commercially available from sources such as SunBio, Seoul, South Korea. PEG compounds may be difunctional or monofunctional, such as methoxy-PEG (mPEG). Activated derivatives of linear and branched PEGs are available in a variety of molecular weights. As used herein, the term “derivatized PEG(s)” or “PEG derivative(s)” means any polyethylene glycol molecule that has been altered with either addition of functional groups, chemical entities, or addition of other PEG groups to provide branches from a linear molecule. Such derivatized PEGs can be used for conjugation with biologically active compounds, preparation of polymer grafts, or other functions provided by the derivatizing molecule.

In another exemplary embodiment, the derivatized PEG is a thiol-derivatized PEG, or sulfhydryl-selective PEG. Branched, forked or linear PEGs can be used as the PEG backbone that has a molecular weight range of 5,000 to 40,000 daltons. In certain exemplary embodiments, the thiol-derivatized PEG is derived from a PEG with maleimide functional group to which a thiol group can be conjugated. In one exemplary embodiment, the thiolated PEG derivative is methoxy-PEG-maleimide, with a molecular weight of 5,000 to 40,000 daltons.

The stealth agents of the present invention may comprise other PEG-like compounds including, but not limited to, thiolated polyoxypropylene polymers, thiolated block copolymers such as the PLURONICs, which are tri-block copolymers comprising polyoxyethylene/polyoxypropylene/polyoxyethylene blocks. Examples of PLURONICS useful in the current invention include, but are not limited to, the following:

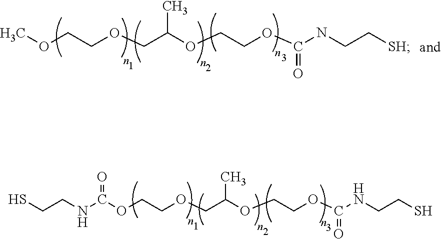

The molecular weight of the PLURONIC block polymer may be from, but not limited to, 1,000 to 100,000 daltons, more preferably between 2,000 and 40,000 daltons.

The polymer blocks are formed by condensation of ethylene oxide and propylene oxide, at elevated temperature and pressure, in the presence of a catalyst. There is some statistical variation in the number of monomer units, which combine to form a polymer chain in each copolymer. The molecular weights given are approximations of the average weight of copolymer molecules in each preparation and are dependent on the assay methodology and calibration standards used. It is to be understood that the blocks of propylene oxide and ethylene oxide do not have to be pure. Small amounts of other materials can be admixed so long as the overall physical chemical properties are not substantially changed. A more detailed discussion of the preparation of these products is found in U.S. Pat. No. 2,674,619, which is incorporated herein by reference in its entirety. (Also see, “A Review of Block Polymer Surfactants”, Schmolka I. R., J. Am. Oil Chemist Soc., 54:110-116 (1977) and Block and Graft Copolymerization, Volume 2, edited by R. J. Ceresa, John Wiley and Sons, New York, 1976

In one exemplary embodiment, the stealth agent comprises polyoxypropylene polymers (POP) that are functionalized, preferably with a thiol group. The preferred molecular weight of the PLURONIC block polymer is between 2,000 and 40,000 daltons. Also included in the present invention are branched polymers, including TETRONIC (PEO/PPO or PEO or PPO) copolymers), branched PEGs and various combinations of the disclosed block polymers. It is understood that linker molecules may be used between the nanoparticle platform surface and the polymer.

In another exemplary embodiment, the stealth agent comprises thiolated poly(vinylpyrrolidone) polymers (PVP) having the following general structure:

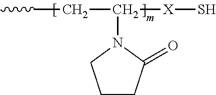

X indicates the site of an optional spacer arm that may be added to the polymer to provide better accessibility of the thiol group to the colloidal metal surface. The spacer arm may be comprised of, but is not limited to, the following propyl groups, amino acids, or polyamino acids. The preferred molecular weight of the PVP polymer is between approximately 1,000 and 100,000 daltons, more preferably between 5,000 and 40,000 daltons.

In yet another exemplary embodiment, the stealth agent is rPEG (Amunix, Mountain View Calif.). As used herein, rPEG generally refers to recombinant PEGylation technology generally involving the genetic fusing of a 300-600 amino acid unstructured protein tail to an existing pharmaceutical protein. Further description of rPEG may be found in United States Patent Publication No. 2008/0039341A1 which is herein incorporated by reference in its entirety.

In another exemplary embodiment, the stealth agent comprises a HES polymer, which is a hydroxyethyl starch (“HES”), a nonionic starch derivative, and is available by Fresenius Kabi, Inc. (Bad Homburg, Germany http://www.fresenius-kabi.com/). HES and HES derivatives may be derivatized and/or thiolated and bound to the colloidal gold nanoparticles.

In another exemplary embodiment, the stealth agent comprises branched aminated PEGs. Branched aminated PEGs suitable for use in the present invention may include, but are not limited to, two, three, four, five, six, seven, and eight branched aminated PEGS. In one exemplary embodiment, the vector compositions comprise colloidal metal sols associated with four arm aminated PEGS. In another exemplary embodiment, the vector compositions comprise colloidal metal sols associated with six arm aminated PEGS.

In another exemplary embodiment, the functionalized polymer may be a PolyPEG® (Warwick Effect Polymers, Ltd., Coventry, United Kingdom). PolyPEG® is a novel pegylating agent for conjugation to therapeutic proteins, peptides and small molecules. PolyPEG®s comprise short PEG chains or “teeth” of varying molecular weight attached to a poly(methacrylate) backbone. The PEG teeth are attached to the poly(methacrylate) backbone via ester linkages, which can degrade over time. Several different chemistries can be used to covalently attached PolyPEG®s to scavenging agents. These include a range of established chemistries for site-specific attachment to free cysteine residues, to the amines on lysine residues, or the terminal amine on biological molecules. Other conjugation chemistries may also be used. The structure of PolyPEG®s can be varied by; (1) the methacrylic backbone which determines the length of the comb; (2) the PEG chain length which determines the quantity of PEG on each tooth of the comb; and (3) the active end-group which determines the site of conjugation between the PolyPEG® and the target biomolecule.

The comb-like architecture of PolyPEG® provides an alternative approach to PEGylation by exploiting the properties of a structure that degrades to small units that are readily excreted over time. This allows their use at high total doses while avoiding potential toxicological problems associated with accumulation of larger molecular weight PEG chains in tissues. PolyPEG®s are similar to conventional PEGs in that they enhance the therapeutic effect of biological molecules by extending their circulatory presence. PolyPEG®s are capable of improving biological activity of certain peptides to a greater extent than convention PEGs. The PolyPEG® molecules can be tailored for a particular requirement for PEGylation of a range of therapeutic molecules.

Nanoparticle Binding Groups

The nanoparticle binding group binds the stealth agent to the nanoparticle platform. The nanoparticle binding group may be bound directly to the core molecule of the stealth agent or may be bound to the stealth agent by a linking molecule. In on example embodiment, the nanoparticle-binding group is a thiol, sulfylhydrl or disulfide group. In another example embodiment, the nanoparticle-binding group is a polyamine, esters, thioesters, thiocarbonates and thiocarbamates.

Exposing Groups

In one example embodiment, the exposing group may comprise higher molecular weight variants of the above listed core molecules. For example the base molecule may be a PEG with a molecular weight of 1 kD and the exposing group may be a PEG with a molecular weight of 20 kD. In certain example embodiments the exposing group has molecular weight at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 times the molecular weight of the core molecule. In certain example embodiments, the higher molecular weight exposing group is linked to the base group by a cleavable linker. The cleavable linker may be an amine linker, an anahydride linker, an imide, thioesters, thiocarbonates and thiocarbamates linker. In certain example embodiment the cleavable linker may be modified to include a functional group that will increase or decrease the rate of cleavage. For example, a functional group could be added to the central N atom of an amide linker to increase or decrease the rate of hydrolysis.

In one example embodiment, the core polymer has a molecular weight of approximately 0.25 kD to approximately 10 kD, approximately 0.25 kD to approximately 9 kD, approximately 0.25 kD to approximately 8 kD, approximately 0.25 kD to approximately 7 kD, approximately 0.25 kD to approximately 6 kD, approximately 0.25 kD to approximately 5 kD, approximately 0.25 kD to approximately 5 kD, approximately 0.25 kD to approximately 4 kD, approximately 0.25 kD to approximately 3 kD, approximately 0.25 to approximately 2 kD, approximately 0.25 to approximately 1 kD, approximately 0.5 kD to approximately 1 kD, approximately 0.5 kD to approximately 2 kD, approximately 0.5 to approximately 3 kD, approximately 0.5 kD to approximately 4 kD, approximately 0.5 kD to approximately 5 kD, approximately 0.5 kD to approximately 5 kD, approximately 0.5 kD to approximately 6 kD, approximately 0.5 kD to approximately 7 kD, approximately 0.5 kD to approximately 8 kD, approximately 0.5 kD to approximately 9 kD, approximately 0.5 kD to approximately 10 kD, from approximately 1 kD to approximately 2 kD, approximately 1 kD to approximately 3 kD, approximately 1 kD to approximately 4 kD, approximately 1 kD to approximately 5 kD, approximately 1 kD to approximately 6 kD, approximately 1 kD to approximately 7 kD, approximately 1 kD to approximately 8 kD, approximately 1 kD to approximately 9 kD, or approximately 1 kD to approximately 10 kD.

In one example embodiment, the exposing group polymer, has a molecular weight from approximately 15 kD to 50 kD, approximately 15 kD to 40 kD, approximately 15 kD to approximately 30 kD, approximately 15 kD to approximately 25 kD, approximately 15 kD to approximately 20 kD, approximately 20 kD to approximately 50 kD, approximately 20 kD to approximately 40 kD, approximately 20 kD to approximately 30 kD, approximately 20 kD to approximately 25 kD, or approximately 25 kD to approximately 30 kD.

In certain example embodiments the bi-functional stealth agent is:

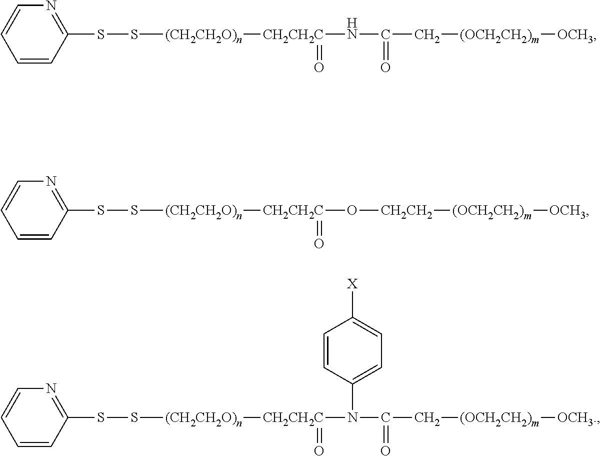

or a combination thereof, where n is a number between 10 and 50, and m is a number between 20 and 1000.

In another example embodiment, the exposing group is a functional group R bound to the core molecule that leaves the nanomedicine with a core negative charge when released from the core molecule. In certain example embodiments the R group is carboxylic acid, an ester, an amide, a thioester, an acyl phosphate, an acyl chloride, thioesters, thiocarbonates, thiocarbamates and acid anhydride.

In certain example embodiments, the bi-functional stealth agent is:

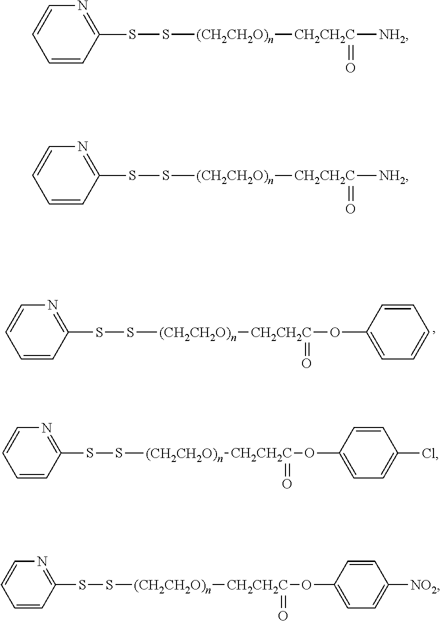

or a combination thereof, where n is a number between 10 and 1000.

### Nanomedicine Medicine Formation

General methods for binding scavenging and stealth agents to nanoparticle platforms may comprise the following steps. A solution of the scavenging agent and stealth agent is formed in a buffer or solvent, such as deionized water (diH₂O). The appropriate buffer or solvent will depend upon the agent to be bound. Determination of the appropriate buffer or solvent for a given agent is within the level of skill of the ordinary artisan. Determining the pH necessary to bind an optimum amount of agent to metal sol is known to those skilled in the art. The amount of agent bound can be determined by quantitative methods for determining proteins, therapeutic agents or detection agents, such as ELISA or spectrophotometric methods. The method of Horisberger (Biol Cellulaire 1979; 36:253-258), (1979), which is incorporated by reference herein, may be used to prepare vector compositions of the present invention. Another suitable method, which may be used in preparation of the vector compositions of the present invention is disclosed in U.S. Patent Application Publication No. 2005/0175584 to Paciotti et al., which is also incorporated herein by reference.

The scavenging agents and stealth agents may be bound directly to the nanoparticle platform or indirectly via integrating molecules. An integrating molecule is a molecule that provides a site for the binding or association of two entities (e.g. a nanoparticle platform and an active agent). Integrating molecules that may be used in the present invention can either be specific or non-specific integrating molecules. The compositions of the present invention can comprise one or more integrating molecules. An example of nonspecific integrating molecules are polycationic molecules such as polylysine or histones that are useful in binding nucleic acids. Polycationic molecules are known to those skilled in the art and include, but are not limited to, polylysine, protamine sulfate, histones or asialoglycoproteins. The present invention also contemplates the use of synthetic molecules that provide for binding one or more entities to the nanoparticle platforms. Specific integrating molecules comprise any members of binding pairs that can be used in the present invention. Such binding pairs are known to those skilled in the art and include, but are not limited to, antibody-antigen pairs, enzyme-substrate pairs, receptor-ligand pairs, and streptavidin-biotin. In addition to such known binding pairs, novel binding pairs may be specifically designed. A characteristic of binding pairs is the binding between the two members of the binding pair. Another desired characteristic of the binding partners is that one member of the pair is capable of binding or being bound to one or more of an agent or a targeting molecule, and the other member of the pair is capable of binding to the nanoparticle platform.

Where integrating molecules are employed in the present invention, the binding pH and saturation level of the integrating molecule is also considered in preparing the compositions. For example, where the integrating molecule is a member of a binding pair, such as streptavidin-biotin, the binding pH for streptavidin or biotin is determined and the concentration of the streptavidin or biotin bound can also determined.

In embodiments where an integrating molecule is employed, the integrating molecule is bound to, admixed or associated with the metal sol. The agent may be bound to, admixed or associated with the integrating molecule prior to the binding, admixing or associating of the integrating molecule with the metal, or may be bound, admixed or associated after the binding of the integrating molecule to the metal.

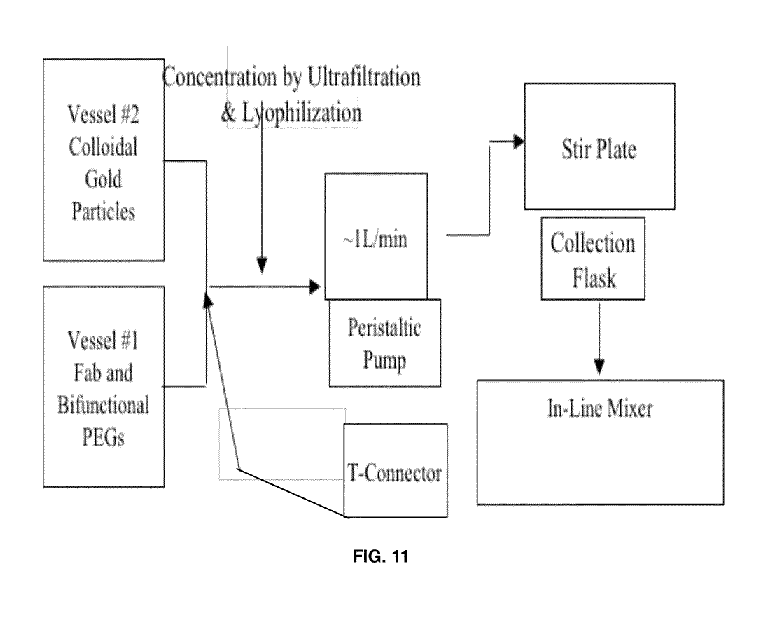

One method of binding an agent to a nanoparticle platform may comprise the following steps, though for clarity purposes only, the exemplary method disclosed refers to binding TNF, to colloidal gold. An apparatus was used that allows interaction between the particles in the colloidal gold sol and TNF in a protein solution. A schematic representation of the apparatus is shown in FIG. 11. This apparatus maximizes the interaction of unbound colloidal gold particles with the protein to be bound, TNF, by reducing the mixing chamber to a small volume. This apparatus enables the interaction of large volumes of gold sols with large volumes of TNF to occur in the small volume of a T connector. In contrast, adding a small volume of protein to a large volume of colloidal gold particles is not a preferred method to ensure uniform protein binding to the gold particles. Nor is the opposite method of adding small volumes of colloidal gold to a large volume of protein. Physically, the colloidal gold particles and the protein, TNF are forced into a T-connector by a single peristaltic pump that draws the colloidal gold particles and the TNF protein from two large reservoirs. To further ensure proper mixing, an in-line mixer is placed immediately down stream of the T-connector. The mixer vigorously mixes the colloidal gold particles with TNF, both of which are flowing through the connector at a preferable flow rate of approximately 1 L/min.

Prior to mixing with the agent, the pH of the gold sol is adjusted to pH 8-9 using 1 M NaOH. Highly purified, lyophilized recombinant human TNF is reconstituted and diluted in 3 mM Tris. Before adding either the sol or TNF to their respective reservoirs, the tubing connecting the containers to the T-connector is clamped shut. Equal volumes of colloidal gold sol and the TNF solution are added to the appropriate reservoirs. Preferred concentrations of agent in the solution range from approximately 0.01 to 15 μg/ml, and can be altered depending on the ratio of the agent to metal sol particles. Preferred concentrations of TNF in the solution range from 0.5 to 4 μg/ml and the most preferred concentration of TNF for the TNF-colloidal gold composition is 0.5 μg/ml.

Once the solutions are properly loaded into their respective reservoirs, the peristaltic pump is turned on, drawing the agent solution and the colloidal gold solution into the T-connector, through the in-line mixer, through the peristaltic pump and into a collection flask. The mixed solution is stirred in the collection flask for an additional hour of incubation.

### Methods of Use and Administration

Described herein are methods for eliminating toxic agents from subjects exposed to the toxic agents. In one example embodiment, the method comprises administration of the compositions described above. The compositions may be administered in formulations suitable for oral, rectal, transdermal, ophthalmic (including intravitreal and intracameral), nasal, topical (including buccal and sublingual), vaginal, or parenteral (including subcutaneous, intramuscular, intravenous, intradermal, intratracheal, and epidural).

In one example embodiment, the composition is administered after exposure to a biological toxin. In another example embodiment, the composition is administered to a high-risk individual, such as a solider, prior to potential exposure to a biological weapon containing the biological toxin. The composition may be administered as a single dose or multiple doses delivered subsequently or contemporaneously. The nanomedicine in each administration may have the same or different residence times in circulation. For example, a first administration of a first nanomedicine with a first circulatory residence time may be made intravenously, and a second administration of a second nanomedicine with second circulatory residence time may be made, the second circulatory residence time being longer than the first circulatory residence time. In the above example, the purpose of the first administration may be to rapidly decrease the amount of toxic agent that has entered the circulation and the second administration may be administered to capture residual toxic agent still in circulation and in tissues. In embodiments where the nanomedicine is administered prior to exposure, the nanomedicine may be further formulated in a microparticle for extended release of the nanomedicine. For example, the nanomedicine may be incorporated into a biocompatible microparticles such as, but not limited to, microparticles made from alginate, methyl methacrylate or PLGA.

The formulations may conveniently be presented in unit dosage form and may be prepared by conventional pharmaceutical techniques. Pharmaceutical formulation compositions are made by bringing into association the metal sol vectors and the pharmaceutical carrier(s) or excipient(s). In general, the formulations are prepared by uniformly and intimately bringing into association the compositions with liquid carriers or finely divided solid carriers or both, and then, if necessary, shaping the product.

This invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLES

### Example 1

### Gold Nanoparticle Synthesis and Characterization

Manufacturing colloidal gold nanoparticles involves the reduction of chloroauric acid (Au⁺³, HAuCl₄) to neutral gold (Au⁰) by agents such as sodium citrate. The reactants, a 4% gold chloride solution and a 1% sodium citrate solution (wt/wt) are made in deionized H₂O (DIH₂O). Particle synthesis is initiated by heating 8 L of DIH₂O to a rolling boil in the reflux apparatus shown in FIG. 11. Subsequently, 20 mL of the 4% gold chloride solution is added through one port in the apparatus. The solution is brought to a boil and kept under reflux during the addition of 320 mL of the sodium citrate solution. After particle synthesis, the sol is cooled to room temperature, filtered through a 0.22μ, nitrocellulose filter, and stored at room temperature until use.

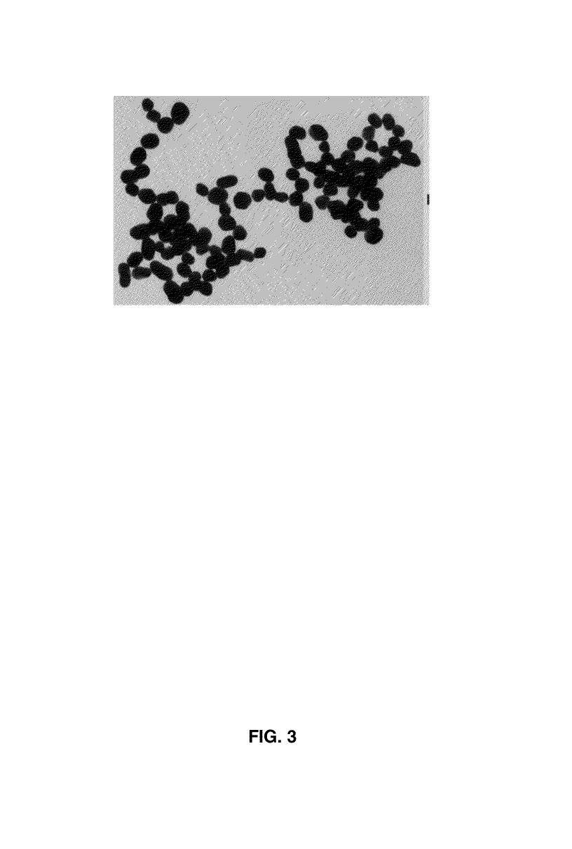

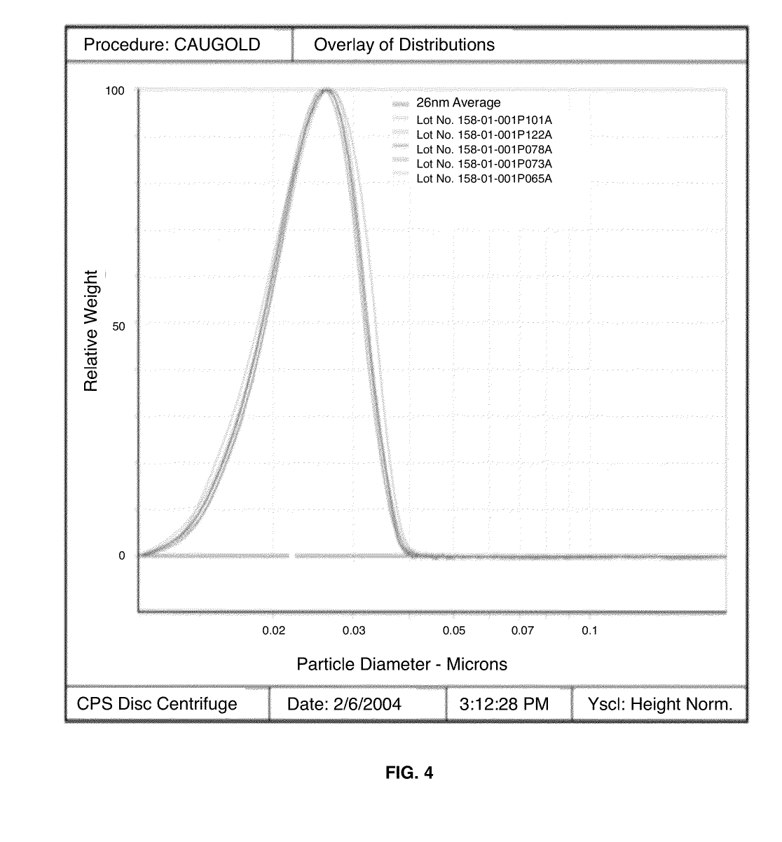

Particle size is determined by three techniques: transmission electron microscopy (TEM; FIG. 3), differential centrifugal sedimentation (FIG. 4) and dynamic light scattering. Electron micrographs are used to physically measure the size of the colloidal gold particles and determine the homogeneity of their shapes. Differential centrifugal sedimentation (DCS), measures particle size as an integral of the time required for the colloidal gold particles to traverse a sucrose density gradient created in a disc centrifuge. DSC only measures core particle size (the size of the gold nanoparticle). In contrast, dynamic light scattering (DLS) is used to measure hydrodynamic volume of the naked gold particles, as well as the nanomedicines prior to and after release of the exposing groups. Both DLS and DCS methods use calibrated reference particles as standards to estimate the size of the colloidal gold preparation. The nanoparticles, as well as the various nanomedicines, are also interrogated for net surface charge by measuring zeta potential.

The procedure outlined above results in the reproducible manufacture of 27 nm particles of colloidal gold. The particles, shown in FIG. 3, exhibit a mean diameter of 27±5 nm, with 95% of the particles having a diameter between 17 and 32 nm. The data presented in FIG. 4 show the reproducibility of the particle manufacturing process.

A fully validated quantitative RP-HPLC method has been developed for determining the concentration of a 20 kD PEG-THIOL. This procedure will be modified to detect the various bi-functional PEGs considered for inclusion in the nanomedicines. Very briefly, following chromatographic separation, the individual bi-functional PEGs are detected by evaporative dynamic light scattering detection (ELSD).

Multiplexed competitive enzyme-linked immunoassays will be used to measure either the seven isoforms of botulinum toxins or the various serotypes of dengue virus in blood and tissues. These assays use pathogen-specific rabbit polyclonal antibodies that are spotted onto distinct sections of each well in a 96-well ELISA plate. Each antibody is used to establish a competitive binding reaction between the pathogen in the sample or standard and a fluorescent form of the same pathogen. Alternatively, the fluorescent signal may be replaced with a pathogen coupled to a quantum dot (QD). The limited number of binding sites that are coated in each well establish a competitive binding reaction between fluorescently labeled pathogen and unlabeled pathogen in the samples or standards. In this reaction, as the concentration of the pathogen present in a sample or standard increases, less of the fluorescent or QD form of the pathogen binds to the coated antibody. Illuminating the wells at the appropriate wavelengths detects the fluorescently (or QD-labeled) labeled pathogen. Thus, as the concentration of pathogen in the sample increases less fluorescence is detected.

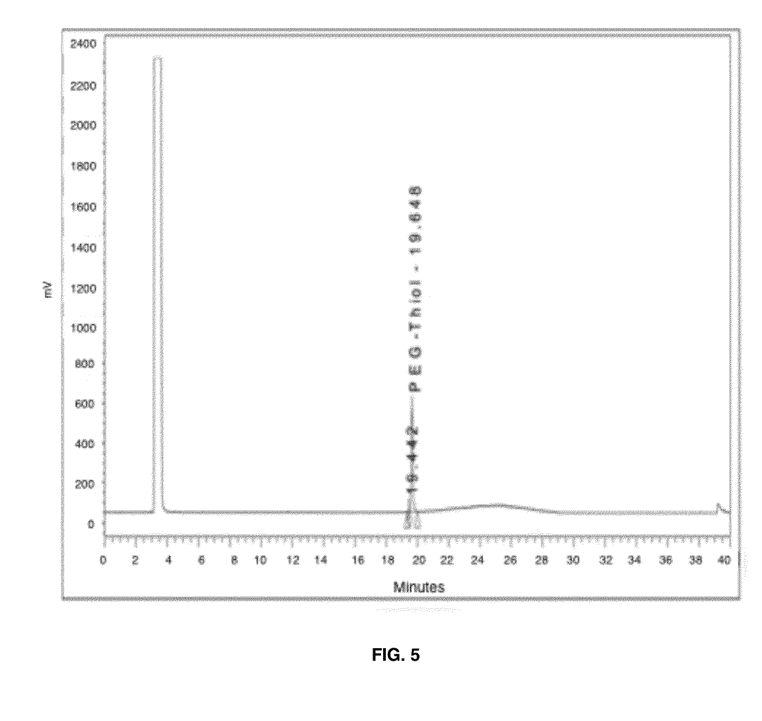

Shown in FIG. 5 is a typical RP-HPLC chromatogram of PEG-THIOL detected by ELSD. The method exhibits linearity and reproducibility over the dose range of 30 to 200 μg/mL. Although changes in the retention time are anticipated, this method is applicable to quantify the intact THIOL-PEG² and THIOL-PEG-R polymers. This method will also be adapted to the track the PEG-molecules following the release of their respective exposing groups.

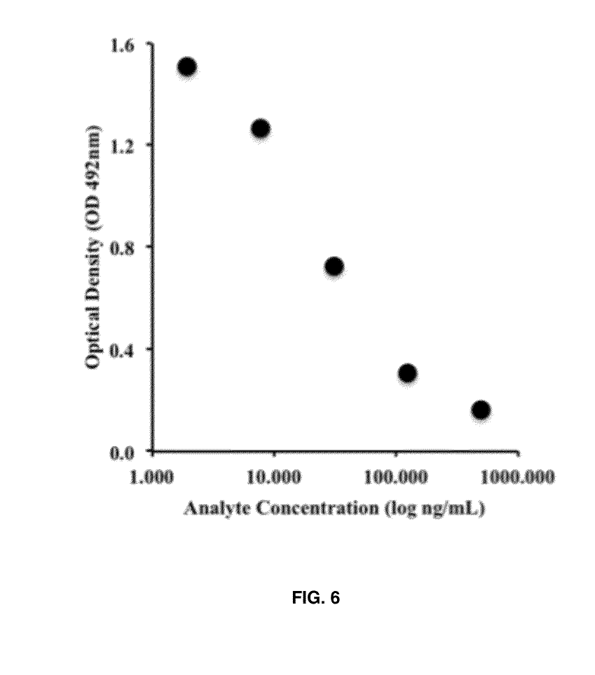

Shown in FIG. 6 is a standard curve for a competitive ELISA using polyclonal antibodies and a colorimetric, rather than a fluorescent, detection system. The data show a clear inverse relationship between the amount of signal detected and the concentration of the analyte. By multiplexing the assay a single well would be able to detect all isoforms/serotypes of the pathogens.

### Example 2

### Gold Nanoparticle Synthesis and Characterization

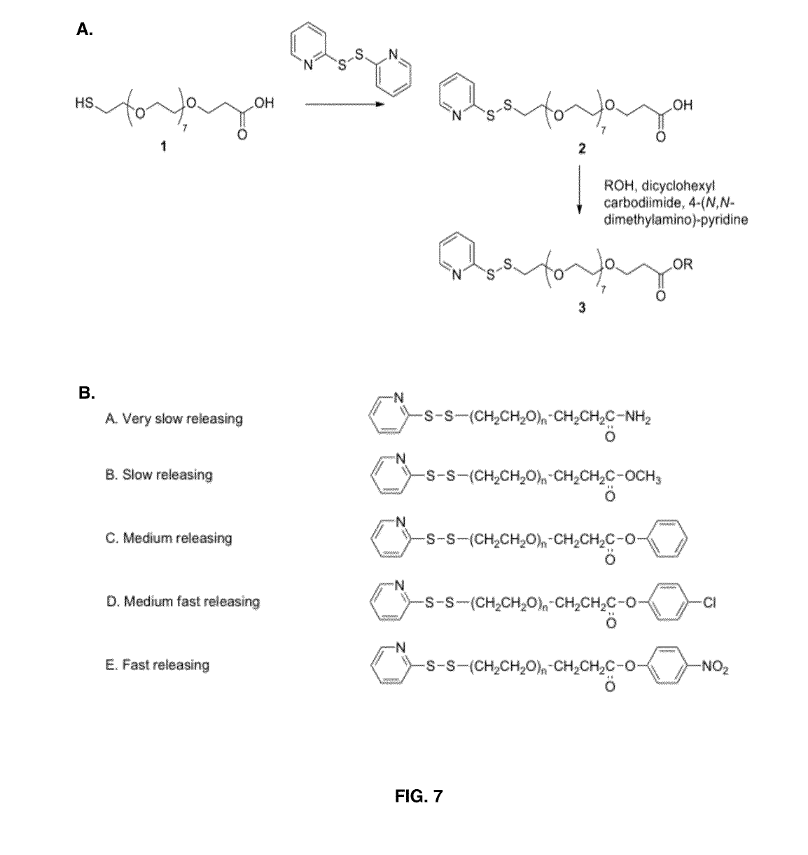

Shown in FIG. 7A is the chemical synthesis of the THIOL-PEG-R polymer which generates the anionic particle in vivo. Briefly, the synthesis begins with the THIOL-PEG-Acid (1) precursor, which is modified at the thiol end with a pyridyl disulfide group to yield the intermediate shown in (2). Subsequently, a variety of functional groups are added to the OH end of the polymer using carbodiimide derivative to generate the final end products shown in FIG. 7B.

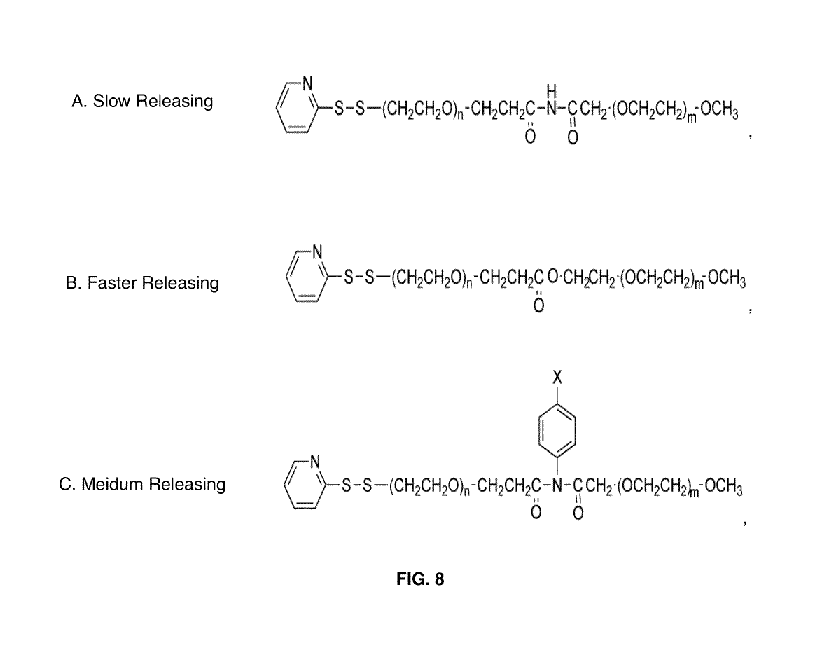

A similar approach is used to generate the THIOL-PEG² polymers described and show in FIG. 8.

An alternative linking chemistry to develop bi-functional polymers involves starting with a commercially available bi-functional THIOL-PEG-Acid polymer. Briefly, the carboxylic acid group on the polymer is oxidized to an aldehyde, which is then chemically coupled to cystamine through one of its free amino groups. Subsequently, a similar reaction is conducted to couple the remaining free amine group to a second molecule of PEG. The end product is THIOL-PEG-cystamine-PEG, which releases the distal PEG molecule by reduction of the disulfide bond.

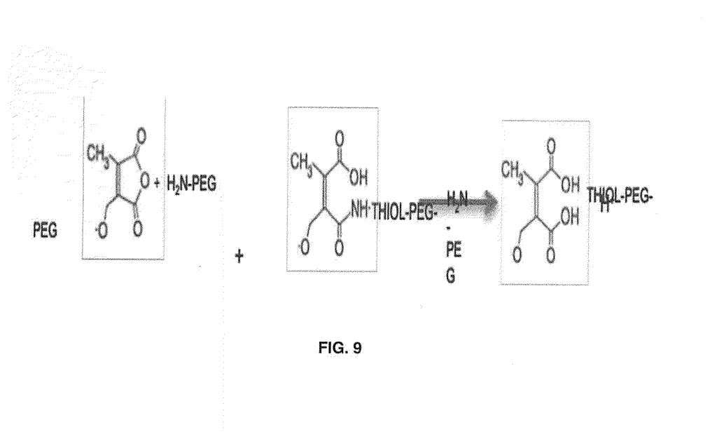

Another possible alternative for developing the THIOL-PEG² polymer utilizes the commercially available starting reagent, THIOL-PEG-maleic anhydride. In this strategy the THIOL-PEG-maleic anhydride polymer is grafted onto a molecule of amine-PEG through the formation of an amide bond. As shown in FIG. 9 the PEG-amine is released under slightly acid conditions, and this reaction is facilitated by the presence of the carboxyl group and the double bond.

### Example 3

### Confirming Release of the Exposing Groups

Once synthesized each polymer is tested to determine the time course required for complete hydrolytic release of the exposing groups. For these studies, each polymer is incubated in a hydrolysis buffer and the release of the exposing groups is monitored by SDS-PAGE analysis of the cleaved polymers. The PEG bands are visualized using a barium iodide stain to track the changes in electrophoretic mobility of each polymer during hydrolytic release of the group.

The release of the exposing group induces different physical changes in the THIOL-PEG2 and THIOL-PEG-R polymers that may easily be tracked by SDS-PAGE. Recall that the THIOL-PEG² polymer consists of two molecules of PEG that are attached to each other through an amine linker. Upon hydrolysis the cleaved polymer would generate two different sized molecules of PEG: a relatively small polymer containing the disulfide group and a larger polymer that is the moiety that creates the water shield around the polymer. The separation of the core polymer into 2 fragments is readily detected by staining of the gels with barium iodine.

The hydrolytic release of the exposing group from the THIOL-PEG-R polymer results in the generation of a free carboxylate group on the distal end (i.e., away from the disulfide group) of the polymer. The increase in net negative charge is monitored by either the HPLC method described above or by gel electrophoresis.

Determining the relative rates for releasing the exposing group allows us to choose the optimal polymer that addresses the clinical sequelae of each bioterror attack. For example, a medium to fast (hours to a few days) releasing polymer may be more appropriate in developing an anti-botulinum nanomedicine, whereas the anti-dengue nanomedicine may require that the polymers provide coverage over much longer period of time. Furthermore, having different rates of release also allows development nanomedicines that address the anticipated differences in time course expected with different routes of exposure. For example, an aerosolized botulinum attack may have a different time course than one in which the toxin is ingested.

Time-course studies determine when the various exposing groups dissociate from the gold nanoparticle-bound bi-functional PEG polymers. These studies are conducted using a series of PEGylated gold nanoparticles manufactured with the various forms of the bi-functional PEGs. Once manufactured the PEGylated gold nanoparticles are incubated with plasma for increasing amounts of time and at designated time points, samples are collected, centrifuged and processed as described below.

For the THIOL-PEG² based nanoparticles the supernatant is removed and the nanoparticle pellet reconstituted with PBS. The particles are washed and re-centrifuged several times and divided into two equal aliquots. The first aliquot is analyzed by dynamic light scattering to determine the anticipated changes in the hydrodynamic volume as the particle bound-THIOL-PEG2 polymer sheds its external PEG segment. The second aliquot of the nanoparticle pellet is treated with beta-mercaptoethanol to release any blood borne proteins that remain associated with the nanoparticles. Following a final centrifugation (to remove the precipitated nanoparticles) the supernatant is analyzed by 2D gel electrophoresis to determine whether particle opsinization occurred.

It is to be understood that this invention is not limited to the particular combinations, methods, and materials disclosed herein as such combinations, methods, and materials may vary somewhat. It is also to be understood that the terminology employed herein is used for the purpose of describing particular embodiments only and is not intended to be limiting.

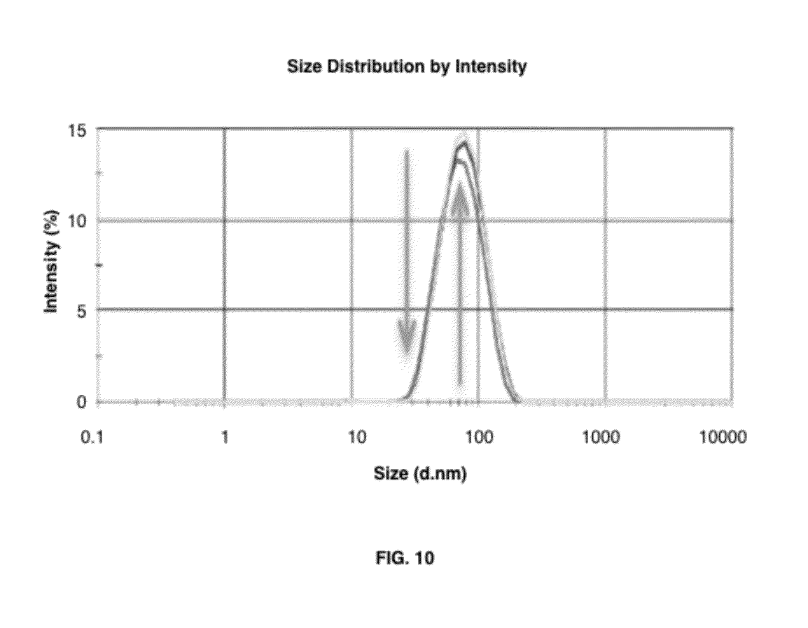

FIG. 10 shows two DLS scans of three different PEGylated gold nanoparticle preparations. These data clearly show that the PEG-mediate hydration of the gold nanoparticles increases the apparent diameter of the nanoparticle from its 27 nm core size as determined by DCS (highlighted by the down arrow) to approximately 80 nm (highlighted by the up arrow).

In a very simple model one could assume that as the PEG shield is lost hydrodynamic volume would decrease, measuring somewhere between the core diameter and the diameter of the fully hydrated particle. Yet as shown in FIG. 10 the loss of the PEG-shield may also induce a layering of blood borne opsins that may actually increase the apparent particle size. Dobrovolskaia show that fully PEGylated gold nanoparticles exhibit very little interaction with blood proteins as very few proteins are detected in a 2D gel. Dobrovolskaia et al. Mol Phar. (2008), 5:487-495. Conversely, unPEGylated gold nanoparticles undergo rapid opsinization in blood as evidenced by the large number of serum proteins bound to the particles' surface. Similarly, we anticipate two possible outcomes with the release of the exposing group from the THIOL-PEG-R based nanoparticles. First, the generation of a net negative charge may induce opsinization as seen in previous work where anionic gold particles coated with protein, not a polymer, were opsinized and taken up by Kupffer cells. Massive targeting of liposomes, surface-modified with anionized albumins, to hepatic endothelial cells demonstrated that anionic particles do traffic to the scavenger receptor. Kamps et al. P.N.A.S. USA: 94, 11681-116845. Another possible outcome is that once the exposing group is released that the nanomedicine has a net negative charge and is taken up by the scavenger receptor. To test this hypothesis THIOL-PEG-R nanoparticles are added to plasma. Subsequently, the particles are washed and centrifuged. The gold nanoparticle pellet is then reconstituted and analyzed for surface charge by zeta potential and protein coating, as described above.

### Example 4

### Generation of Human Anti-Toxic Agent Monoclonal Antibodies

CytImmune has developed a simple technology for the in vitro production of fully monoclonal antibodies against a variety of self and non-self antigens. The technology fully replicates the in vivo human antibody response as evidenced by the de novo synthesis of human antibodies against a broad spectrum of protein antigens. Furthermore, the antibody responses are not recall responses as the profile of antibodies produced is similar to the typical in vivo antibody responses reported following immunization (i.e., antigen-specific IgM antibodies are produced first, followed by class switching to antigen-specific IgG or, depending on the application, antigen-specific IgA or IgD antibodies).

Typically, a single buffy coat, a unique fraction of human whole blood containing white blood cells, including antigen presenting cells, (i.e., monocytes) and antibody secreting cells (i.e., B-cells), is purchased from a vendor licensed to collect and sell human blood. Upon receipt, the white blood cells are enriched from the buffy coat by centrifugation on Ficoll-Hypaque so that following centrifugation the white blood cells form a layer on the surface of the boundary between the blood plasma and the Ficoll-Hypaque solution.

The enriched white blood cells are collected, centrifuged and washed several times in heparinized saline. Subsequently, B-cells and monocytes are sequentially purified using murine monoclonal antibodies that are linked to magnetic beads specific for the B-cell marker, CD19, or the monocyte specific marker, CD14. Upon isolation each cell fraction (i.e., the B-cells and monocytes) are characterized for purity by FACS analysis and only preparations showing greater than 95% purity are used for in vitro immunization. The purified B-cells and monocytes are subsequently mixed at a ratio of 1 to 5 and a total of 500 B-cells are cultured in a 6-well plate in 2.5 mL of RPMI with 10% FBS. These cells form the base for the in vitro immunization culture described below. As outlined in the introduction there are seven botulinum toxins that maybe weaponized for use in a bioterror attack. Although it is certainly possible to generate unique human mAbs that are specific for each toxin, our initial goal is to develop a single human mAb that binds an amino acid sequence shared by all seven toxins. These three amino acids (amino acid nomenclature, ALN) occur from amino acids 657-659, which is shared by all seven isoforms of the toxin. Moreover, toxins B and G share an additional amino acid sequence homology in position 660 (ALNV), while five of the toxins (A, E, F, D and C) have the amino acid sequence ALNI in common. Sharma, S. K., Basavanna, U. and Shukla, H. D. 2010. Protein Domain Analysis of C. Botulinum Type A Neurotoxin and Its Relationship with Other Botulinum Serotypes. Toxins. 2: 1-9.

With respect to dengue virus, a recombinant form of the envelope protein DENV E or an attenuated form of the whole virus serotype is used to immunize the B-cell/monocyte co-culture.

To increase the likelihood of generating antibodies to the consensus sequence of botulinum toxin these peptides are bound to 8-10 nm (not 27 nm) colloidal gold particles. Unlike larger proteins such as the intact botulinum toxins or the DENV E protein smaller peptides require the gold nanoparticle to make them immunogenic. Based on prior experience with the small peptide, EGF, that is approximately 5 kD, only colloidal gold-bound EGF was immunogenic in our in vitro culture system. As previously described, a gold-peptide immunocomplex is created to drive antigen uptake and processing by dendritic cells. For example, the consensus sequence is bound to 8-10 nm gold particles that also contain both TNFα and IFNγ. The rationale for having the pathogen bound to these cytokine-containing gold nanoparticles is that these cytokine-containing particles continually drive the uptake and processing of the consensus sequence gold immunocomplex to the phagocytic pathway.

If peptide antigens fail to generate antibodies capable of recognizing all seven isoforms of the botulinum toxins, then a cocktail of heat-denatured toxins is used to immunize a new set of B-cell/monocyte cultures. To generate fully human mAbs against dengue virus, human white blood cell cultures are immunized with a heat denatured form of either the DENV E protein or whole virus (if in fact the protein vaccination fails).

B-cell-monocyte co-cultures are immunized with 1-10 μg of either the gold-bound immunocomplex or the heat-denatured forms of each pathogen. Each culture is incubated for approximately 2-3 weeks. During this initial period the monocytes differentiate into antigen presenting cells by taking up the protein antigens and breaking them down into peptide fragments. These fragments are then presented, along with a variety of co-stimulatory molecules, on the surface of the monocytes and induce the resting B-cells to mount a pathogen-specific antibody response. In response to this immunogenic stimuli the B-cells start secreting an IgM class of antibodies, supporting our hypothesis of a de novo antibody response (i.e., not a recall response). However, IgM antibodies are not suitable for the proposed nanomedicines as they are usually low affinity antibodies. In vivo, such antibody producing B-cells undergo a process known as class switching, which adds functionality to the antibodies being produced as well as increases their affinity.

Class switching can be induced with the addition of growth factors whose role in the antibody maturation process have been clearly identified. Two weeks after immunization a cytokine cocktail consisting of CD40L, IL-2 and IL-21 is added to the cells. One week after the addition of this cocktail, an aliquot of the incubation media is collected and analyzed, by standard ELISA techniques, to confirm that pathogen specific antibodies are being produced. Botulinum toxin-immunized samples are screened to ensure that the antibodies that are generated at this early stage recognize all seven isoforms of the toxin. Similarly, the dengue-immunized blood samples are screened to ensure that the antibodies recognize the whole virus, the DENV E protein, as well as the amino acid sequence involved in viral uptake. At this stage of the response antibodies that are being generated are diverse (polyclonal) and most likely detect their respective pathogen at multiple sites.

Following a second two-week incubation the antibody producing B-cells are immortalized by fusing them with a cancer cell, a myeloma. The polymer, polyethylene glycol (PEG), is used to physically fuse the cell membranes of K6/H6 heteromyeloma cells with the membranes of the cultured cells. After fusion the cells are cultured in 96-well plates using RPMI supplemented with the toxin hypozanthine aminopterin thymidine (HAT). HAT is a toxin, which can only be metabolized by fused cells. All other fused cells (i.e., B-cell-B-cell, B-cell-monocyte, monocyte-monocyte) die during this selection process. The end-result of the fusion is the generation of a hybrid cell, termed a hybridoma that continually grows, like a cancer cell, producing antibodies, like an immunized B-cell. Two weeks after fusion the plates are visually scanned to identify wells in which growing cells are present. 100 μl of the culture supernatant is removed from these wells and analyzed, by ELISA, for the presence of pathogen specific antibodies.

During the continued growth, the hybridomas act as mini-bioreactors that secrete antibodies. Consequently, the antibody from each clone is screened to ensure that it meets the minimum criteria for recognition of each pathogen. For example, the botulinum toxin-specific clones are screened to identify clones that secrete antibodies recognizing all seven isoforms of the toxin. Similarly, the dengue virus clones are screened to ensure that they recognize the entire virus, as well as the DENV E protein.

Under the most ideal conditions, a hybridoma secretes a single antibody. However, more likely a cluster of hybridomas will contain two or more unique cells secreting different forms of the antibody, and, as is often the case in hybridoma development, a single 96-well may contain more than one cluster. Thus, to identify and characterize a single form of the antibody, all clones secreting appropriate antibodies (i.e., antibodies that recognize the 7 isoforms of the botulinum toxin) are scaled up from a 96-well tissue culture plate to a 25 mL tissue culture flask.

Subsequently, the scaled cells are collected by centrifugation and re-cultured in two 96-well plates to undergo serial limiting dilutions. The cells are cultured until the cells plated in the red-boxed area become confluent (i.e., covering the bottom of the well). At this point media from these cultures are once again tested to identify the clones with the fewest number of cells that are still secreting antibodies and meeting the selection criteria (i.e., antibody against botulinum toxin that recognizes all 7 isoforms). Take for example the well that's highlighted with a red star as representing the highest limiting dilution that generates a positive antibody signal against all 7 isoform of botulinum toxin while the wells highlighted with the black stars do not. After confirmation the cells in the red-starred well are collected and scaled once again for a second round of limiting dilutions to identify a single-cell clone that secretes a single form of the anti-botulinum toxin antibody (a monoclonal antibody).

This single hybridoma is grown in large quantities and the resultant antibody purified from the tissue culture media by standard chromatographic techniques. Once purified to homogeneity the pathogen specific human mAb is characterized to identify chemical characteristics (i.e., class and subclass) as well as affinity (how strongly) it binds its target antigen. Based on previous experience, minimally 20-35 individual clones for each pathogen are generated and characterized for a lead candidate for developing each nanomedicine.

Only the IgG_{1&3} class of monoclonal antibodies is possible candidate components for developing the nanomedicine as they possess the highest affinities in the IgG class of antibodies.

Determining the antibody affinity is measured by surface plasmon resonance (SPR). For this study, purified monoclonal antibody is bound to the surface of a gold SPR component of a BiaCore 30000 to establish a baseline SPR measure. Subsequently, the antigen is passed over the surface of the antibody-coated chip and the change in SPR is monitored. Once binding is complete buffer is passed over the antibody-antigen complex to initiate dissociation of the antibody-antigen complex. The release kinetics, a measure of its affinity, is made as an integral of the time for the SPR measure to baseline. Thus, the longer it takes for the antibody-antigen complex to dissociate the higher the affinity. Antibodies with affinities in the 10⁻¹⁰ to 10⁻¹¹ M are selected to ensure that once the antibody binds the pathogen, the complex does not dissociate.

The ability of the antibody to bind the pathogen and prevent it from accessing its cellular recognition site is known as neutralization. At this time we hypothesize that affinity rather than pathogen neutralization is a more critical selection criteria.

Prior to developing the biodefense nanomedicine the purified antibody is processed to facilitate its binding to the gold nanoparticle. Passing the whole antibody preparation through a papain column fragments the antibody into two distinct fractions. Papain digestion yields a heavy chain fragment, which is discarded. The second fraction, known as the antigen-binding fraction, represents the portion of the antibody that binds and immobilizes the various pathogens onto the particles' surface. Papain digestion also may facilitate the binding of the Fab to the gold nanoparticles, since upon digestion disulfide groups may become available to form the dative covalent bond between the Fab and the gold nanoparticle. Recall, free thiol groups or disulfide bonds on proteins and gold atoms on the surface of the nanoparticle form dative covalent bonds. The dative covalent bond has the energy equivalence of a C—C bond and as we have noted with our cancer nanomedicines the bond allows particle bound moieties to remain attached to the particle surface while in the circulation.

### Example 5

### Nanomedicine Development

Colloidal gold nanoparticles remain in suspension by their mutual electrostatic repulsion due to a net negative charge on the particles' surface. Cations present in salt solutions negate this charge repulsion and cause these “naked particles” to agglomerate and eventually precipitate out of solution. The precipitation of the particles is easily documented as the color of the particles changes from red to black. Binding of proteins or other stabilizing agents to the particles' surface maintains the sol state by blocking the salt-induced precipitation of the colloidal gold particles. To facilitate the binding of the Fabs to the particle surface requires the determination of the pH binding optimum.

The binding of proteins to colloidal gold nanoparticles is dependent on the pH of the colloidal gold and the protein solutions. To determine the optimal pH for binding antibody fragments to the colloidal gold particles, the pH of 2 ml aliquots of 27 nm colloidal gold sol is adjusted, as measured on pH strips, from pH 5 to 11 using 1N NaOH. The Fab fragments are diluted to a concentration of 1 mg/ml and further diluted to 100 μg/ml in 3 mM TRIS base pH 8. 100 μL of stock is added to the seven aliquots of pH-adjusted colloidal gold and incubated for 15 min. Following this incubation, 100 μl of a 10% NaCl solution is added to each of the aliquots to induce particle precipitation. The pH binding optimum is defined as the pH that allows the protein to bind to the colloidal gold particles to prevent their precipitation by salt, as described above. Shown in FIG. 14 is an example of a pH optimum binding study of an ideal protein. In this case, the pH optimum is between pH 4 and 6. Unlike the Fabs, these studies need not be performed for the thiolated bi-functional PEGs as the presence of the thiol group will allow binding to occur over a broad pH range.

As previously described, the dative covalent bond is a bond formed between free thiols/disulfides and the gold surface. Having the energy equivalence of a C—C bond these dative bonds ensure that the constituents of the gold nanomedicines remain attached to the surface of the gold nanoparticle once injected into the circulation. Although ionic and hydrophobic bonding is also possible many such groups (i.e., multiple ionic or multiple hydrophobic bonds) are required to achieve the same strength of bonding as a single dative covalent bond.

The disulfide groups present on the Fabs may facilitate the formation of the dative covalent bonds. For these studies, colloidal gold bound Fab particles are generated at the pH binding optimum. Then to determine which mechanism mediates the binding of these proteins to the nanoparticles the preparations are incubated in buffers that interfere with each type of binding. For example, to disrupt ionic interactions, Fab-gold nanoparticles are incubated in a high ionic strength buffer, whereas to disrupt hydrophobic interactions the preparations are incubated in detergent-based buffers. To demonstrate dative covalent binding, sample preparations are incubated in buffers that contain the reducing agent β-mercaptoethanol. If the data from these studies point to dative binding then the next set of studies in which the binding capacity of the gold particles is determined for their respective Fabs is undertaken. In the event that mixed mode binding is observed additional thiol groups are added to the carboxylic end of Fab to favor dative covalent binding.

The goal of this experiment is to estimate the binding capacity of the gold nanoparticles for each of the Fabs. Based on the data obtained from the pH binding study, the pH of 27 nm colloidal gold sol is adjusted to the pH binding optimum for the botulinum and dengue Fabs. Each sol is divided into 1 ml aliquots to which increasing amounts of the Fabs are added to their respective gold preparations and incubated. After binding for 15 min, the samples are centrifuged at 7,500 rpm for 15 min, and the resultant colloidal gold pellets and supernatants separated. The pellet fraction is reconstituted to its original 1 mL volume and subsequently treated with β-mercaptoenthanol to release the particle bound material. Both the supernatant and pellet samples are analyzed by quantitative ion exchange HPLC using standard techniques.

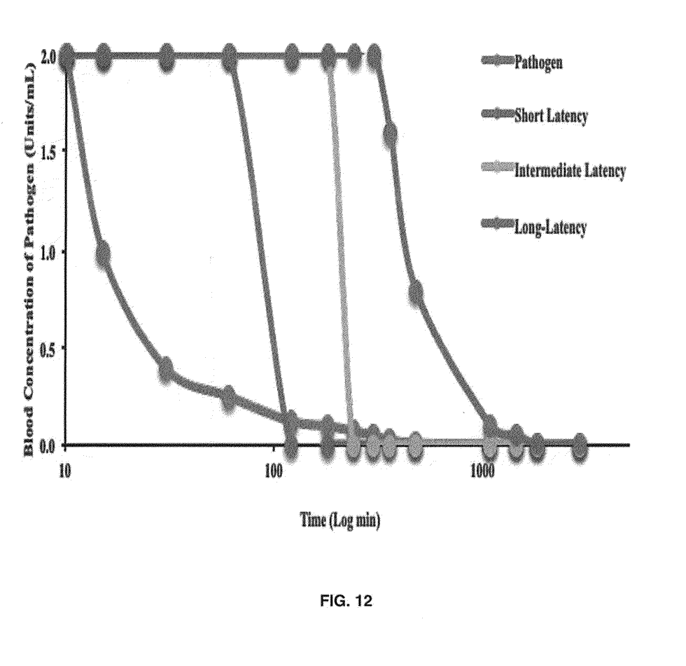

The biodefense nanomedicines are manufactured using the process, shown in FIG. 12. Briefly, two glass containers are filled with either the colloidal gold nanoparticles or a solution containing the bi-functional PEGs. To initiate binding, the solutions are brought into contact with each other through a Y-connector fitted with in-line mixers. This process is both reproducible and extremely flexible in generating formulary variants of the nanomedicines by simply altering the concentration of either the polymer or Fab in the reactant vessel. After binding, a series of stabilizers are added to the solution, which is then concentrated by ultrafiltration and lyophilized. Throughout the manufacturing process, in-process samples as well as final product samples are collected and analyzed by for RP-HPLC/ELSD for the polymer and the light chain specific ELISA to detect the specific Fab.

### Example 6

### Characterization of Nanomedicines

Primary neurons isolated from the medial basal hypothalamus are isolated as described by Skaper or purchased from Caygen Inc. Skaper S D, Adler R, Varon S. 1979. A Procedure for Purifying Neuron-Like Cells in Cultures from Central Nervous Tissue with a Defined Medium. Dev Neurosci. 2:233-237. The cells are plated on poly-1-lysine plates and are cultured for up to 2 weeks prior to use. On the day of the study the cells are incubated with increasing doses of botulinum toxin A or the same doses that were previously incubated with the nanomedicine.

The cells are incubated with either native or nanomedicine-complexed toxins for several hours to allow the toxin to interact with the cells and degrade the SNAP proteins regulation neurotransmitter release. Subsequently, the cells are harvested, counted, lysed and the intracellular SNAP detected by Western blot analysis following SDS-PAGE electrophoresis. The density of the resultant bands is quantified by densitometry and normalized for the number of cells harvested.

The goal of the following studies is to determine the ability of the dengue-targeted nanomedicine to block the debilitating effect of an initial dengue infection and the life-threatening side effects mediating dengue hemorrhagic fever. The key to achieving both goals is to rapidly clear the virus from the body during the initial infection to prevent the initial immune response to the virus.

Recall that current hypotheses support that the antibodies generated during the first infection greatly enhance the uptake of the virus by its host cells during re-infection Nielsen D. G. Virology Journal, (2009), 6:211. The goal is to optimize the release of the exposing group to prevent activation of the immune system. As an additional benchmark the delivery of the nanomedicine is tailored to match anticipated conditions in the field of operation.

To test the ability of the dengue targeted nanomedicine to block infection, the monocyte/B-cell co-culture, originally described in the in vitro generation of serotype-specific dengue fully human mAbs, is adapted as a model for infection. For these studies the human B-cells and monocytes are prepared and co-cultured as described above. Subsequently, increasing amounts of one of the serotypes of dengue is added to the culture alone or complexed to the nanomedicine. The cells are incubated for a period of one to two weeks upon which the culture supernatant is tested for the presence of human-anti-dengue antibodies.

Additionally, to model the clearance of the nanomedicine complexed dengue a 3-well flow through system is used. The first well is used for the addition of the nanomedicine or pathogen preparations. In addition human or mouse Kupffer cells are cultured in the second well while the B-cell/monocyte co-culture is cultured in the third well. Subsequently, various forms of the dengue-targeted nanomedicine are incubated with either human plasma or in buffer for increasing periods of time for the release of the exposing group. Upon the generation of either an opsinized or anionic nanomedicine-pathogen complex the preparations is added to the first well and allowed to flow through the Kupffer cell to the B-cell monocyte co-culture. The effluent from the B-cell monocyte cultured is completely removed collected and stored. Fresh culture media is added to all the wells and a slow and continuous flow of fresh media is passed over the culture system for an additional two weeks during which time the production of human antibodies as an output signal is monitored.

The impact of the botulinum targeted nanomedicine on the pharmacokinetics (PK) and tissue distribution of botulinum toxin is evaluated using three experimental protocols. The goal of the first experiment is to develop a baseline pharmacokinetic profile for the toxin by intravenously injecting 2 μg of the A isoform of the toxin into naïve mice. Then, at selected time points groups of animals are bled through the retro-orbital sinus and the animals sacrificed by CO₂ inhalation. The animals are sacrificed at 5, 15, 30, 60, 120 180, 240, 360, 480, 1080, 1440, 1800 and 2880 minutes. Subsequently, the entire lung, kidney, brain, liver and spleen of each animal are harvested at each time point and flash frozen for analysis.

On the day of the analysis the organs are homogenized in saline containing PMSF and bacitracin. Subsequently the blood and tissue homogenates are analyzed for botulinum content using the multiplexed competitive ELISA described above. To develop the PK profile the WinNonLin program is used to calculate Cmax (The maximal concentration achieved in blood), Volume of Distribution (Volume into which the toxin distributes), T_{1/2} (A measure of how long the toxin is present in the circulation), Area Under Curve (a measure of exposure) and Clearance (a measure of how rapidly the toxin is eliminated).

The botulinum-targeted nanomedicine is tested in both preventative and interventional models that differ in the time of, and possibly route of, administration of the nanomedicine. In the preventative model, mice are intravenously injected with the A isoform of the toxin and immediately treated with an intravenous injection of the various botulinum targeted nanomedicines. The interventional studies differ in that the time for injecting the nanomedicine is increased from 30 minutes to several hours after toxin administration.

In a final study, a modified interventional study is conducted in which intoxicated animals receive, in addition to an IV injection, a subcutaneous injection of the nanomedicine. With a SC injection the nanomedicine is able to scavenge toxin at tissues sites and re-enter the circulation through the lymphatic system. One of the more challenging aspects of developing dengue specific therapies is the lack of appropriate animal models for testing the putative therapy. For example, mice, rats and non-human primates exhibit various degrees of pathogenesis in response to infection with any of the dengue serotypes. For example, Bente has shown that only severely immunocompromised mice exhibit a pathological response to infections while infection of wild type mice and non-human primates dengue infection results in attenuation of the virus and loss of infectivity. Bente, D. A. and Rebeca Rico-Hess, R. 2006. Models of dengue virus infection Drug Discov Today Dis Models. 3: 97-103.

To address this challenge transgenic mice in which the murine genes controlling the immune response have been replaced with the human equivalence is used. These transgenic mice are normally used for generating fully human antibody therapeutics and thus represent an excellent model for mimicking dengue infection in humans as they will generate similar T- and B-cell responses to viral infection.

Two sets of studies are conducted to model a primary and secondary infection. In an initial study the time course for infection and the generation of the immune response by following the production of human antibodies following infection with dengue is established. These data then provide the framework to develop the planed preventative or interventional studies with the nanomedicine. Briefly, to demonstrate the ability of the nanomedicine to prevent a primary infection naïve transgenic mice are infected with one of the dengue serotypes, alone or following its being complexed with the nanomedicine. Heparinized blood is collected and analyzed for a panel of markers to indicate the activation state of B- and T-cells, while the plasma is tested for the presence of dengue specific antibodies. For one of the interventional studies the transgenic mice are infected with dengue and following a 2-6 period are treated with the serotype specific nanomedicine and the immune response tracked as described above.

Shown in FIG. 12 are hypothetical pharmacokinetic profiles comparing the blood levels of the native pathogen with those of the pathogen bound to the nanomedicines. Note that these representations are exaggerated to illustrate the latency between the nanomedicine injection and the release of its respective exposing group.

The blue line represents the typical pharmacokinetic profile of the pathogen as it enters the circulation and then distributes to its target tissue where it will exert its effects. However, in the presence of the nanomedicine we anticipate that the blood levels may not decrease overtime but rather stay at some steady state level. This is consistent with our hypothesis that the nanomedicine binds, using the particle bound hu-mAb, to the pathogen, maintaining it in the circulation and preventing it from reaching its target site. Subsequently once the exposing group is released a rapid elimination of the pathogen nanomedicine complex from the circulation as complex is taken up by the MPS is anticipated. The actual time that the complex remains in the circulation will be dictated by the latency of the exposing group release from the bi-functional PEG. However as noted in the graphs once the exposing group is released a precipitous drop in the blood levels is expected as the nanomedicine-complexed-pathogen is rapidly removed by the Kupffer cells.

The expected outcome is preventing the onset of respiratory paralysis leading to progressive morbidity and mortality. Targeting the toxin in both blood and tissues is expected to greatly reduce or eliminate respiratory paralysis. This outcome might also be improved by modulating the time-delay between the administration of the nanomedicines and its release of the exposing group.

The in vitro model described above is expected to be predictive of the in vivo behavior of the dengue-targeted nanomedicine. The ability of the nanomedicine to directly couple with the virus, or indirectly, through uptake by the Kupffer cells, prevents the activation of the immune response while simultaneously clearing the pathogen. In vitro, these data are seen in the lack of an antibody response from the B-cell/monocyte co-culture. Similarly, we anticipate that eliminating the virus by the Kupffer cells not only blocks infection, but also blocks the activation of the immune response. Consequently, upon re-infection transgenic mice receiving the nanomedicine therapy might not exhibit the augmented pathogenicity typically noted in cases of re-exposure.

We claim:

1. A composition comprising, a nanoparticle platform, a bi-functional stealth agent bound to the nanoparticle platform, and a scavenging agent that binds a target agent bound to the nanoparticle platform.

2. The composition of claim 1, wherein the nanoparticle is colloidal gold.

3. The composition of claim 1, wherein the target agent is a bacterial toxin, or a viral coat protein.

4. The composition of claim 1, wherein the bacterial toxin is a botulinum toxin.

5. The composition of claim 1, wherein the viral coat protein is a dengue virus coat protein.

6. The composition of claim 1, wherein the scavenging agent is a monoclonal human antibody.

7. The composition of claim 1, wherein the bi-functional stealth agent comprises a nanoparticle binding group, a core polymer, a cleavable linker, and an exposing group polymer.

8. The composition of claim 7, wherein the core polymer is a thiolated PEG with a molecular weight of approximately 0.25 kD to approximately 1 kDA.

9. The composition of claim 7, wherein the exposing group polymer is a PEG group with a molecular weight of approximately 1 kD to 40 kD.

10. The composition of claim 7, wherein the exposing group is one or more of the following; 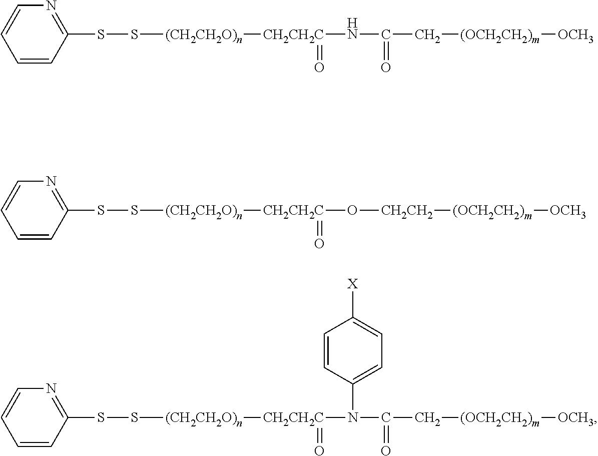
and
wherein n is between 10 and 50 and m is between 20 and 1000.

11. The composition of claim 7, wherein the cleavable linker is an amine linker, an anhydride linker or an imide linker.

12. The composition of claim 1, wherein the bi-functional stealth agent comprises a thiol-PEG-maleic anhydride bound to an amine PEG.

13. The composition of claim 1, wherein the bi-functional stealth agent comprises a nanoparticle binding group, a core polymer, and an exposing group.

14. The composition of claim 13, wherein the core polymer is a thiolated PEG with a molecular weight of approximately 0.25 to 1 kD.

15. The composition of claim 13, wherein the exposing group comprises a pyridyl disulfide group bound to a free terminus of the nanoparticle binding group.

16. The composition of claim 13, wherein the bi-functional stealth agent is one or more of the following: 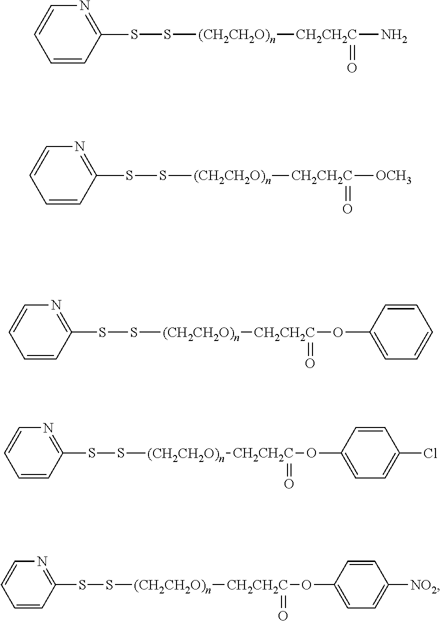
and
wherein n is between 10 and 1000.

17. A method for capturing and eliminating pathogens from subjects exposed thereto, the method comprising administering a composition to a subject exposed to a pathogen, the composition comprising a nanoparticle platform, a bi-functional stealth agent, and a scavenging agent that binds the pathogen or a component of the pathogen.

18. The method of claim 17, wherein the pathogen or a component of the pathogen is a botulinum toxin or a dengue virus coat protein.

19. The method of claim 18, wherein the bi-functional stealth agent comprises a nanoparticle binding group and an exposing group, or a nanoparticle binding group, a cleavable linker, and an exposing group.

20. The method of claim 17, wherein the composition is encapsulated in or bound to a microparticle.
